SEQUENCE LISTING (6 sequences)

SEQ ID NO: 1 - DNA, 21 bp, Artificial Sequence
  Description of Artificial Sequence DNA encoding in single letter code PEPTIDE
cctgaacctactattgatgaa

SEQ ID NO: 2 - DNA, 21 bp, Artificial Sequence
  Description of Artificial Sequence  DNA encoding in single letter code SLMBAA
tctttgatggccgcggcagcg

SEQ ID NO: 3 - DNA, 30 bp, Artificial Sequence
  Description of Artificial Sequence   DNA encoding in single letter code PLANTCILIC
cctcttgctaatacttgtatacttctttgt

SEQ ID NO: 4 - DNA, 21 bp, Artificial Sequence
  Description of Artificial Sequence   DNA encoding in single letter code USASCLAB
ttgtctgcttcttgtcttgcc

SEQ ID NO: 5 - DNA, 21 bp, Artificial Sequence
  Description of Artificial Sequence   DNA encoding DIAGNOS
gatattgctggtaatataact

SEQ ID NO: 6 - DNA, 9 bp, Artificial Sequence
  Description of Artificial Sequence   DNA encoding 293
tgccaggac

(19) United States
(12) Patent Application Publication
(10) Pub. No.: US 2005/0282169 A1
(43) Pub. Date: Dec. 22, 2005

(54) Signatory sequences

(76) Inventors: Allen C. Turner, Salt Lake City, UT (US); Johan Renes, Soest (NL)

Correspondence Address: TRASK BRITT, P.O. BOX 2550, SALT LAKE CITY, UT, 84110, US

(21) Appl. No.: 10/767,911

(22) Filed: Jan. 29, 2004

(51) Int. Cl. C12Q 1/68; A01K 67/00; C12P 21/06; A01H 1/00; C12N 15/82

(52) U.S. Cl. 435/6; 435/69.1; 435/325; 435/419; 435/320.1; 435/468; 530/350; 800/8; 800/278; 380/59

(57) ABSTRACT

The invention includes a method of labeling a biological polymer involving including within the polymer a series of monomers that encode a source of origin or other useful information regarding the biological polymer. Preferably, the biological polymer is DNA, and the series of monomers spell the name of the entity creating the biological polymer using the single letter codes of amino acids corresponding to codons encoded by the DNA.

### TECHNICAL FIELD

The invention relates generally to biotechnology and more particularly to the field of molecular biology. More in particular it relates to recombinant DNA technology. In particular, the invention relates to providing biological materials such as cell, vectors, cosmids, plasmids, microorganisms, and/or bio-organic polymers such as polypeptides and other nucleic acids with an identifier, thereby enabling identification of the originator, owner or other person or entity associated with the bio-organic polymer and/or the host cell, organism, microorganism, vector or vehicle comprising the bio-organic polymer or polymers.

### BACKGROUND

In modern molecular biology significant amounts of new bio-organic polymers are produced by life science laboratories. In particular, many vectors of many different origins comprising different regulatory elements and/or different markers and/or different genes of interest (including resistance/amplification genes) are made basically on a daily basis. These vectors are often available in different host cells and/or microorganisms.

Many researchers exchange materials such as vectors and/or cells on a regular basis. This is often done on a good faith basis (without any contracts such as Material Transfer Agreements). Be that good or bad, it thus becomes very difficult to trace the origin of certain materials. Traceability is very important for a number of reasons. Many researchers, for instance, are not actually the owners of the results of their research. The institute or company they work for typically has the rights to all results and materials produced by the researcher. If these institutes and/or companies have intellectual property rights to materials produced it is of vital importance to them that they know what happens to these materials. It is also of vital importance regarding possible liabilities.

In certain instances, it becomes of great importance to be able to determine the source of origin of a biological material or product such as a plasmid, bacteria, cell, virus, or transgenic animal or plant.

For instance, a plasmid encoding human growth hormone disappears from a lab refrigerator in a university during a New Year's Eve party and turns up in a biotechnology company's laboratory. Significant expenses in legal fees and investigations are spent determining the actual source of the plasmid.

In another case, anthrax is spread in an act of terrorism through the U.S. Mails. Again, significant time and effort is expended in trying to determine the source of origin of the biological product, i.e., the laboratory from which the bacteria originated.

It would be an improvement in the art if biological products were marked in such as a way as to readily determine their source of origin. In this manner, the bona fide origin or the lack of a bona fide origin of biological materials can be easily determined.

### DISCLOSURE OF THE INVENTION

The present invention provides a way of making materials in modern molecular biology identifiable so that they or their progeny (if the materials are capable of replication/reproduction) can be traced and/or identified as belonging to or originating from a certain entity.

Thus, the invention provides in one embodiment a method for identifying a person or entity associated with the biological material (e.g., an owner, originator, licensor, and/or licensee) in a nucleic acid sequence or an amino acid sequence comprising providing a unique combination of building blocks in the sequence, the combination of building blocks being representative of the owner or originator. The sequence of building blocks is referred to herein as an identifier.

An amino acid sequence identifier can be represented by nucleic acid sequences providing codons that correspond to the amino acids in the sequence. Such a nucleic acid sequence (representative of the amino acid sequence) need not be expressed and indeed, may be arranged so that it is not expressed. By providing the identifier in codon language, however, it is possible to provide more identifying characters (syllables and/or numbers) than based on nucleotides alone. Table B herein gives one possible way of providing codons representative of all English language letters and numbers. The nucleic acid sequences according to the invention can be produced in any suitable manner. Oligonucleotides may be commercially purchased, for example, from the NAPS Unit of the University of British Columbia in Vancouver, Canada.

Alternatively, nucleotides may be synthesized using known techniques and equipment such as an Applied Biosystems 380B DNA synthesizer, Applied Biosystems 392 DNA/RNA synthesizer, an Applied Biosystems 394 DNA/RNA synthesizer, an Applied Biosystems 3900 High Throughput DNA synthesizer, and/or a Biolytic Lab Performance Cycleaver 12 (for bulk ammonia cleavage) or an automated system.

In one embodiment, the automated system for synthesizing the nucleic acids sequences includes software for automatically (or by user selection) incorporating the identifier sequence into other synthesized nucleic acid. In a preferred embodiment, this is achieved by a simple conversion of the word provided (e.g., the identifier sequence) automatically translating the word into codons (e.g., using Table B herein).

Typically, oligonucleotides are synthesized from 3′ to 5′ on a solid support CPG or polystyrene resin using phosphoramidite chemistry. Following synthesis, each oligonucleotide is cleaved from the solid support with concentrated ammonium hydroxide, and then incubated at an appropriate temperature overnight. The deprotected oligonucleotide is then desalted by, for example, ammonia/butanol extraction and then dried down in a labeled tube.

Alternatively, the signature sequence can be incorporated into the sequence during the entire sequence's construction.

Introduction of a nucleic acid sequence in a larger sequence, such as a vector, a plasmid, a cosmid, or a genome of a cell, the genome of a microorganism and/or the genome of an organelle can be achieved by methods well known in the art. By providing suitable flanking sequences, a sequence or sequences may be introduced using restriction enzymes. By providing suitable complementary sequences, a sequence or sequences can be introduced by homologous recombination and by providing suitable priming sequences a sequence or sequences can be introduced by primer extension and/or amplification techniques.

Any known method of introduction can be used with a strong preference for methods that allow control of the site of introduction. When such control is provided, the signatory sequence can be introduced into sites known not to affect the desired properties of the biological material. In one embodiment, for example, when the biological cells are eukaryotic or prokaryotic cells, the synthesized nucleic acid is circularized, associated with, for example, calcium phosphate, and may be taken up by the cells for marking the cells.

A particularly preferred embodiment of the invention involves providing identifiers through the process of homologous recombination. (See, e.g., EP 0 505 500 B1, published Jul. 30, 1997, the contents of which are incorporated by this reference). A typical construct for homologous recombination is depicted in FIG. 1, wherein A and B are targeting regions (complementary to sequences in the nucleic acid to be provided with an identifier), C is an identifier sequence, D is a positive selection marker (such as neo), optionally with an amplifier function (DHFR provides both functions), E is a negative selection marker (such as HSV-Tk). The presence of D and E is optional, but preferred, due to the ease of screening for successful introduction of the construct (D) and selecting out random integrants (E). In a process according to the invention, the construct of FIG. 1 is introduced into a cell, or contacted with a nucleic acid to be provided with an identifier in any suitable manner. Conditions are chosen which allow for homologous recombination to occur and successful integrants by homologous recombination are selected by culturing in appropriate media. Of course, this process can be carried out with more than one identifier introduction by homologous recombination simultaneously, or sequentially.

After synthesis, the nucleic acid can be incorporated into a plasmid or into an organism's genome. In one embodiment, the signature sequence can be flanked by a chosen sequence that is relatively easy to find by computerized nucleic acid analysis.

In one embodiment, there are no start or stop codons associated with the signature sequence. This prevents expression of the corresponding protein that might interfere with its therapeutic utility. Furthermore, preferably there are no known restriction sites associated with the signature sequence.

It will not always be necessary to introduce a sequence into a nucleic acid molecule or cell, or microorganism that needs to be identifiable. Sometimes, it may be sufficient to designate nucleotides and/or codons already present as identifiers, preferably such nucleotides and or codons are in non-coding areas. However, these designated nucleotides/codons should be unique and easily determined in the identifiable biological material by a relatively simple assay method such as amplification (e.g., by polymerase chain reaction or “PCR”). Simple assayability is typically provided when the signatory sequence (be it already present or introduced) is provided in one stretch. Thus in one preferred embodiment the signatory sequence is a contiguous sequence.

It is a preferred embodiment of the invention to provide a signatory sequence that is representative of the owner or the originator in the sense that it provides a word, words, or combination of characters that is associated with the company and/or institute and/or person who is owner and/or originator. A trade name or trade or service mark expressed in codon language would be perfectly suitable. In single stranded sequences it is possible to “hide” the word or combination of characters by providing it in complementary codons. The originator/owner has the option to provide a clear reference to the originator/owner or to provide a signatory sequence that is only apparent to the owner/originator.

If the biological material to be identified is a vector, plasmid, or the like, the identifier can very suitably be the code used to identify the plasmid (e.g., pbr 2232) or the like, possibly in combination with a word (trademark, service mark, and/or trade name) identifying the originator. A cell line can be provided with the name of the cell line (e.g., A549, 911, and so forth) again optionally together with a word identifying the owner/originator. The principle of identification should be clear to the person skilled in the art based on these two examples.

Once introduced or designated, the signatory sequence is preferably not removed too easily to avoid tampering. Cleavage sites are preferably not present in the vicinity of the signatory sequence. Multiple signatory sequences at different sites with different flanking regions are also helpful to prevent unwanted removal.

In one embodiment, a database of such “signature” sequences is maintained for entities using, administering, governing, or regulating the system. The administrator of the database preferably chooses code letters corresponding to non-traditional selections of codes (e.g., numerals). The database keeps a record of the various particular sources of origin used by an entity taking advantage of the invention. Preferably, the database can be accessed “on-line” (e.g., by the Internet or modem hook-up) so that entities utilizing the system can input new entries and update old ones. Preferably the database software conducts a search of the remainder of the database to ensure that the particular entity or any other one has not already used a duplicate signature sequence. A chronological record of the database is preferably kept for historic, evidentiary, and fraud prevention reasons. Also, to prevent fraud and to enhance the security of the system, the system is preferably encrypted and requires a key or password for access. An entity using the system will preferably be able only to see its own database entries, and not those of other entities using the system.

Such a database is preferably administered electronically, with the use of commercially available computer equipment that, once being made aware of the invention, will be readily recognized and chosen by those of skill in the database and Internet arts. For instance, the database can be kept on a personal computer having a central processing unit, memory, adequate storage space (e.g., a multigigabyte hard drive), and, preferably, broadband access to the Internet. An Internet website for user access can be hosted by any of various commercial website providers.

Encryption can be by one of the various systems currently commercially available or improvements and other modifications thereof as can password entry into the website.

Optical or other inalterable back-up systems are preferred.

In one embodiment; the particular signature sequence is registered with the Library of Congress as a copyright registration and with the U.S. Patent & Trademark Office as a trademark. This is done so in the event that the plasmid is stolen and used by an unscrupulous competitor, claims for both copyright and trademark infringement might also be made.

In one embodiment, the invention includes a method for identifying an owner and/or originator in a nucleic acid sequence or an amino acid sequence, the method comprising providing an identifier which is a unique combination of building blocks in the nucleic acid sequence or an amino acid sequence, the combination of building blocks identifying the owner and/or originator. In the method the building blocks are preferably amino acids or nucleotides/nucleosides and the identifier is the trade name or a trademark of the owner, licensee, licensor, and/or originator.

In another embodiment, the invention includes a nucleic acid sequence comprising an identifier. The identifier comprises a selected combination of nucleotides/nucleosides, the selected combination of nucleotides/nucleosides identifying the owner or originator (e.g., a contiguous sequence of nucleosides/nucleotides). The selected combination is preferably unique and corresponds to a trade name and/or trademark of the owner and/or originator. Preferably, the identifier is essentially free of nuclease susceptibility.

The invention also includes a cell or microorganism comprising the nucleic acid sequence, preferably integrated into the cell's genome and/or the genome of at least one of the cell's organelles.

In another embodiment, the invention includes a polypeptide comprising an identifier, the identifier being a unique combination of amino acids representative of an owner or originator of the polypeptide. Preferably, such an identifier is essentially free of protease susceptibility.

The invention also includes a method for determining the origin of a biological material, comprising subjecting the biological material to an assay capable of determining the presence of an identifier in the biological material. In such a method, the identifier is preferably a nucleic acid sequence, and the assay comprises at least one probe or primer capable of hybridizing to the nucleic acid sequence (e.g., the assay comprises materials necessary for a nucleic acid amplification method such as PCR or NASBA for the sequence).

The invention also includes a plasmid of the type including a nucleic acid sequence of interest, wherein the improvement comprises choosing and integrating, into the plasmid, an indicator of source of origin (e.g., a nucleic acid sequence encoding a peptide having a single letter code sequence spelling a name) to identify an owner or originator the plasmid. Preferably, the nucleic acid sequence encodes a peptide having a single letter code sequence spelling a name, the sequence not configured for expression. The invention also includes a cell comprising such a plasmid and a non-human transgenic organism (e.g., a plant or animal) containing such a cell. Preferably, the indicator of origin is a nucleic acid sequence encoding a name using codons encoding a letter of the alphabet or a number.

The invention also includes a method of marking a biological polymer (e.g., DNA, RNA, polysaccharide, or polypeptide) comprising monomers, wherein the method comprises including, as a portion of the monomers, a series of monomers encoding a source of origin.

The invention also includes a method of identifying a first biological polymer, the method comprising incorporating into the first biological polymer (e.g., DNA, RNA, and polypeptide) an indicator of source of origin comprising a second biological polymer encoding the source of origin; analyzing the first biological polymer to determine the first biological polymer's sequence, including the sequence of the second biological polymer; and reading the sequence of the second biological polymer to determine the source of origin. Preferably, the second biological polymer encodes the source of origin of the first biological polymer by corresponding monomers in the polymer to a letter of the alphabet or a number.

The invention includes a method for marking a biological polymer with a source of origin, the method comprising determining a code of monomers, the monomers being of biological origin, wherein at least one monomer corresponds to at least one alphanumeric character; translating an indicator of source of origin for an entity into a series of the monomers; and incorporating the series of monomers into a biological polymer made by or for the entity. Preferably, the source of origin encodes the entity's name.

### BRIEF DESCRIPTION OF THE FIGURE

FIG. 1 depicts a construct for homologous recombination. A and B are targeting regions (complementary to sequences in the nucleic acid to be provided with an identifier), C is an identifier sequence, D is a positive selection marker (such as neo), optionally with an amplifier function (DHFR provides both functions), and E is a negative selection marker (e.g., HSV-Tk).

### BEST MODE OF THE INVENTION

In one preferred embodiment, the genetic code serves as the basis for the system. The single letter codes (“SLC”), amino acid names, three letter codes (“TLC”), and corresponding DNA codon or codons are given here:

TABLE A

| SLC | AMINO ACID | TLC | CODON(S) |
|---|---|---|---|
| A | Alanine | Ala | GCT, GCC, GCA, GCG |
| B | None | — | None |
| C | Cysteine | Cys | TGT, TGC |
| D | Aspartic Acid | Asp | GAT, GAC |
| E | Glutamic Acid | Glu | GAA, GAG |
| F | Phenylalanine | Phe | TTT, TTC |
| G | Glycine | Gly | GGT, GGC, GGA, GGG |
| H | Histidine | His | CAT, CAC |
| I | Isoleucine | Ile | ATT, ATC, ATA |
| J | None | — | None |
| K | Lysine | Lys | AAA, AAG |
| L | Leucine | Leu | CTT, CTC, CTA, CTG, TTA, TTG |
| M | Methionine | Met | ATG |
| N | Asparagine | Asn | AAT, AAC |
| O | None | — | None |
| P | Proline | Pro | CCT, CCC, CCA, CCG |
| Q | Glutamine | Gln | CAA, CAG |
| R | Arginine | Arg | CGT, CGC, CGA, CGG, AGA, AGG |
| S | Serine | Ser | TCT, TCC, TCA, TCG, AGT, AGC |
| T | Threonine | Thr | ACT, ACC, ACA, ACG |
| U | None | — | None |
| V | Valine | Val | GTT, GTC, GTA, GTG |
| W | Tryptophan | Trp | TGG |
| X | None | — | None |
| Y | Tyrosine | Tyr | TAT, TAC |
| Z | None | — | None |

As can be seen, no amino acids correspond to the single letter codes for English alphabet characters B, J, O, U, X, and Z or the numbering system. In such a case, various methods may be used to accommodate the situation. For instance, a particular codon for one amino acid having more than corresponding codon (e.g., alanine, glycine, isoleucine, leucine, proline, arginine, serine, threonine, or tyrosine) can be substitute into the code to correspond to such a letter. For example, “GCC”, which codes for alanine, can be deemed to code for the letter “B”. “J” could be encoded by “AAG”. “ATA”, which codes for isoleucine, can be deemed to correspond to, for example, the letter “O”. “TTG”, which codes for leucine in the genetic code, can be deemed to code for the letter “U” herein. “GTG”, which encodes for valine, could be used for “X”. “AGC”, which encodes serine, could be “Z” in the system.

In one embodiment, the letters can be coded by, for example, doublets, triplets, quadruplets, quintuplets, sextuplets, septuplets, or octuplets. Each doublet (or triplet, quadruplet, etc.) of amino acid would then represent a letter. With such a system, numbers could also easily be incorporated into the source of origin indication identifier and indicate, for example, a particular lab or batch within an organization from which the cell, plasmid, transgenic organism, etc. originated.

Alternatively, numbers could be encoded by one of the extra codons or by other means. For example, the number “0” could be encoded by “GCA”, “1” could be encoded by “GCG”, “2” could be encoded by “TGC”, “3” could be encoded by “GAC”, “4” could be encoded by “GAG”, “5” could be encoded by “TTC”, “6” could be encoded by “GGC”, “7” could be encoded by “GGA”, “8 could be encoded by “GGG”, and “9” could be encoded by “CAC”. Of course, other combinations and permutations could be selected. Also, a single codon could be used to code for a letter in one position and a numeral in another (e.g., if the codon is in the “last” position, it could represent a numeral, while in any other position, it could encode for a letter.

Using such a system, the following chart would result:

TABLE B

| CHARACTER | CODON |
|---|---|
| A | GCT |
| B | GCC |
| C | TGT |
| D | GAT |
| E | GAA |
| F | TTT |
| G | GGT |
| H | CAT |
| I | ATT |
| J | AAG |
| K | AAA |
| L | CTT |
| M | ATG |
| N | AAT |
| O | ATA |
| P | CCT |
| Q | CAA |
| R | CGT |
| S | TCT |
| T | ACT |
| U | TTG |
| V | GTT |
| W | TGG |
| X | GTG |
| Y | TAT |
| Z | AGC |
| 0 | GCA |
| 1 | GCG |
| 2 | TGC |
| 3 | GAC |
| 4 | GAG |
| 5 | TTC |
| 6 | GGC |
| 7 | GGA |
| 8 | GGG |
| 9 | CAC |

Although one set of chosen codes is depicted in Table B herein, other choices may be used (e.g., selecting GCC for the letter “A”, TGC for C, GAC for D, and so forth). The chosen codes need not even correspond to the single letter codes, but they are preferably used for convenience.

The identifier, signature, or signatory sequence is chosen, preferably in some easy to understand format (e.g., the name of the company, university, laboratory, or researcher together with some other indication of origin such as lab number). When the signature sequence utilizes the one-letter codes of the genetic code, the signature sequence is reverse translated into the corresponding nucleic acid sequences encoding the sequence. For instance, the nucleic acid encoding the signature sequence “PEPTIDE” could be CCTGAACCTACTATTGATGAA (SEQ ID NO:1).

When the signature sequence utilizes a nucleic acid sequence as the polymer, for example, a company whose initials spell “CAT” could, for instance, merely have a series of repeating “CATs” incorporated into a plasmid or other nucleic acid sequence.

For RNA viruses, a system using RNA as the polymer can be readily adapted and utilized (e.g., substituting the appropriate RNA for DNA) by, for example, the use of a cDNA or infectious clone.

Another method of providing, for example, primary cells with an identifier is by fusing such a cell with an immortal cell (e.g., a myeloma cell) that has already been provided with an identifier in its genome. Another way of immortalizing is for instance by introducing an adenoviral sequence into the genome, which comprises E1 sequences from adenovirus or Epstein Barr Virus. The identifier of the invention can also be added to such an adenoviral sequence. The same goes for other immortalizing sequences that are introduced into (primary) cells.

In prokaryotes, an embodiment may be used where signatory sequences are included in self-replicating plasmids (episomal) in the prokaryote. By virtue of their self-replication, the signatory sequences will also be found in the progeny.

Again, more than one identifier may be present per self-replicating plasmid(s) or different self-replicating plasmid(s) may carry different identifiers. Also, the identifier may be divided and incorporated into different places in the biological material (e.g., plasmid(s) or genome).

In one embodiment, especially useful in the agricultural market, the invention provides identifiers to modified live vaccines (viral or bacterial). This is a very suitable method to distinguish in a subject (e.g., a mammal) between the presence of a wild-type infection and the presence of vaccine material.

In still another embodiment, especially useful in the diagnostic market, the identifier is expressible and of a size (e.g., >about 8 to 10 amino acids in length) capable having antibodies raised against the modifier (e.g., by the well known process of Kohler & Milstein, the phage display process, or ribosome display process). Such antibodies can be used to detect the identifier. In this embodiment, it is preferred that expression of the identifier be under the control of an inducible promoter.

If there are two or more originators/owners (including licensors/licensees) of a biological material, any of them can have their own identifier within one signatory sequence or they can have separate signatory sequences with their own identifiers.

Sequences of biological materials can be analyzed for the signature sequence using known methods. To prevent accidental “infringements”, the signature sequence can be flanked by a chosen sequence that is relatively easy to find (e.g., sequences encoding the same amino acid multiple times). “BLAST” searching can be utilized.

For detection purposes, a nucleotide may be sequenced by means known to those skilled in the art. For instance, a DNA sequencing service could use Applied Biosystems (Foster City, Calif., USA) instrumentation and chemistries. Such equipment includes an Applied Biosystems PRISM 377XL (64-lane), an Applied Biosystems PRISM 377 (96-lane), a Perkin Elmer DNA Thermalcycler 480 (48-tube), an Applied Biosystem GeneAmp PCR System 9600 (96-well format), an Applied Biosystem GeneAmp PCR System 9700 (96-well format). Such chemistry includes Applied Biosystems BigDye™ v3.1 Terminator Chemistry, Applied Biosystems BigDye™ dGTP Chemistry available for GC-rich templates, and Applied Biosystems dRhodamine Chemistry available for homopolymer regions. Fluorescent dye terminator chemistry may be used to run in the same tube using a standardized thermalcycler program.

Of import to the success of sequencing reactions run is the quality and quantity of sample provided. Contaminated templates yield high background noise and poor, or no, sequence information. Preferably, the template and primer concentrations are measured carefully, as incorrect quantification, whether higher or lower, will cause poor or no sequencing results. It is also vital that the template and primer must be resuspended in water, and not TE buffer, as EDTA will interfere with the ion concentration in sequencing reactions.

Equipment useful for protein/peptide sequencing includes an Applied Biosystems 476A Protein Sequencer, and an Applied Biosystems Procise 494 Protein Sequencer.

N-terminal sequencing of proteins or peptides may be performed on an Applied Biosystems' 476A or Procise 494 automated sequencers using standard gas phase or pulsed liquid Edman chemistry. The 476A and 494 Procise are equipped with an on-line reverse phase HPLC+610A data analysis system. Separation and analysis of the amino acid sequence occurs on the basis of the derivatized amino acids affinity for the stationary phase of the RP-PTH-C18 column packing material.

Protein samples may be enzymatically digested and the resulting peptides are separated by capillary HPLC (“cLC”) and collected onto PVDF membrane using, for example, an Applied Biosystems 173 MicroBlotter. The individual peptides bound to the PVDF are subsequently subjected to N-terminal sequencing analysis using automated protein sequencers.

While the goal of sequencing a protein sample is to identify as many amino acids as possible using the least amount of sample, success can be limited by several factors. One stumbling block to successful sequencing is insufficient amount of material. Usually a 10 pmol sample for 5 amino acids to be identified. Preferably, the minimum number of cycles for sequencing is five. In order to identify a protein stringently, 15-20 residues are commonly used.

Sequencing may be limited by an inability to obtain sufficient amounts of adequately purified protein. Samples should contain one protein component only and reagents which interfere with the sequencing process should be avoided. The presence of contaminants increases the likelihood that ambiguous data will be obtained and the chances of miscalls are greater. Clean samples tend to yield better results and sequence further. Contaminating peptides or proteins contribute to a higher noise level of non-sequence related amino acids.

The invention is further explained with the help of the following illustrative examples.

### EXAMPLES

### Example I

A computer for housing a database of such “signature” or signatory sequences is set-up and maintained for entities using the system. The computer is an IBM compatible computer having a one-gigabyte, INTEL central processing unit, 512 MB RAM, and a 60-gigabyte hard drive. The computer uses a MICROSOFT operating system. The computer has a T-1 line for access to the Internet. The computer uses a commercially available back-up system that records the back-up material onto CDs or other suitable media. It also has a “mirror system” to ensure redundancy and preserve the integrity of the system. Back-up CDs are stored offsite to prevent accidental damage.

The web page is secured with password access and encryption. Providing password protection to the website can be done with the use of readily commercially available software (e.g., a perl CGI script used to manage multiple usernames/passwords for .htaccess/.htpasswd directory protection, such as .htaccess Manager Version 3.3 available from TechnoTrade of Kailua-Kona, Hi., US).

Encryption may be accomplished with commercially available software such as “Pretty Good Privacy” (e.g., PGP Version 6.5.8 that includes PGPnet) available from MIT (MA, US), Network Associates (Santa Clara, Calif., US), and RSA Security (Bedford, Mass., USA). A commercial web site host hosts it.

Database software for use with the invention is readily commercially available (e.g., SQL database from Microsoft, Redland, Wash., US). It keeps a record of the various particular signature sequence used by an entity and the particular biological material into which the signature sequence is incorporated (e.g., a plasmid or plant seed). It is accessible on-line via the Internet connection so that entities utilizing the system can input new entries and update old ones. An entity using the system is only able to see its own database entries, and not those of other entities using the system.

An Internet website can be hosted by any of various commercial website providers.

Optical or other inalterable back-up systems are preferred.

### Example II

State University, in one of its microbiology labs, “MB-101”, develops a plasmid encoding a gene product useful in the treatment of anemia. In the plasmid, going in the sense direction, the following DNA sequence is incorporated by known techniques: TCT TTG ATG GCC GCG GCA GCG ((SEQ ID NO:2) of the accompanying and incorporated by this reference SEQUENCE LISTING). As can be seen, using the one letter code, this sequence would spell “SLMBAAA”, but using the aforementioned substitution of “U” for “L” with respect to TTG and the aforementioned numerical substitutions, actually spells “SUMB101” for State University Microbiology Laboratory 101.

A researcher at State University accesses the database of EXAMPLE I via the Internet, and inputs an identification of the plasmid as well as the sequence of SEQ ID NO:2. Other information can also be input if desired (e.g., position of the marker within the plasmid, relevant dates, researcher names, function of a protein encoded by the plasmid, remainder of the plasmid's sequence, etc.) The database software conducts a search to ensure no one else has used such an identifier, and confirms this to the researcher.

### Example III

A company, “PLANTCO”, which genetically modifies plants finds that by altering a particular nucleotide sequence in a particular plant's genome to an antisense direction increases the production by the plant of a desired metabolite (e.g., a secondary metabolite or an oil) or imparts some other desired property upon the plant (e.g., resistance to an insect pest or herbicide). PLANTCO has a licensee, LIC, which is to market the genetically modified plant pursuant to a license agreement.

Before transferring the genetically modified plants to XYZ, the particular sequence is incorporated into the plant's genome (or the plant's seed). Also incorporated into the plant's genome is nucleotide sequence spelling out PLANTCO's name together with the licensee's name, in this case, CCT CTT GCT AAT ACT TGT ATA CTT CTT TGT (SEQ ID NO:3). As can be seen, this nucleic acid sequence, but for the lack of a start codon, would spell “PLANTCILIC” using the one letter amino acid codes, but using the aforementioned substitution of ATA for “O”, actually spells “PLANTCOLIC” indicating a source of origin for the plant genome, i.e., the PLANTCO's licensee, LIC.

LIC transfers the seeds of the plant in violation of the license agreement existing between PLANTCO and the licensee. Plants having the desired characteristic (e.g., increased secondary metabolite production) begin appearing on the black market. The genome of a plant purchased on the black market is analyzed, and the sequence SEQ ID NO:3 is found identifying the source of the plant.

### Example IV

A disease causing microorganism, for example, a plague causing bacteria is experimented with in a United States government laboratory located in Small City, USA. The bacteria are genetically altered to include a marker plasmid having the following sequence: TTG TCT GCT TCT TGT CTT GCC (SEQ ID NO:4), which using the foregoing code spells “USASCLAB” for USA Small City Laboratory.

A disgruntled employee of the lab removes some of the bacteria from the laboratory, and attempts to mail it to politicians via the U.S. Postal Service. The bacteria are intercepted and analyzed. The marker plasmid is found, and the source of bacteria determined. The disgruntled employee is interviewed, investigated, and arrested.

### Example V

A company, DIAGNOS sells antibody test kits for hepatitis B that include, on the solid phase, a recombinantly produced hepatitis B surface antigen. Black market versions of the test kit are being introduced into the market, which test kits lack the sensitivity and accuracy of DIAGNOS' test kits, but are otherwise a perfect “knock off” with respect to packaging and presentation.

Into the hepatitis B surface protein, DIAGNOS introduces the codons corresponding to the name DIAGNOS, for example, GATATTGCTGGTAATATAACT (SEQ ID NO:5) into the plasmid coding for the HBsAg. The plasmid is taken up into the bacteria expressing the HBsAg with the aid of CaPO₄.

### Example V

For providing identifiers for RNA materials, such as RNA viruses (e.g., for use with modified live vaccines) it is preferred to provide the identifier in a cDNA copy (preferably an infectious clone in the case of RNA viruses). By virtue of the process of transcription, the identifier is present in the corresponding RNA biological sequence.

### Example VI

Homologous recombination example. The purpose of this example is to provide 293 cells with the identifier “293”. In the codon language of Table B, “293” is translated to TGCCACGAC (SEQ ID NO:6).

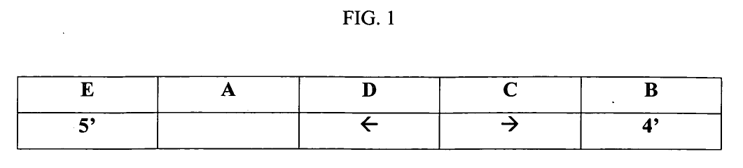

293 cells are cultured in a suitable culture medium. A site for introduction of the identifier is selected. A construct is designed having targeting regions A and B complementary to sequences in the selected site (FIG. 1). The construct further comprises, between the targeting regions, the identifier sequence TGCCAGGAC (SEQ ID NO:6) and the positive selection marker neo under its own promoter, preferably in the opposite direction compared to the identifier and the cell's genome. The construct further comprises a negative selection marker HSV-Tk outside the homologous recombination regions. The construct is part of a plasmid suitable for transmission into 293 cells. The plasmid is transferred into 293 cells using the well-known calcium phosphate precipitation method (Van der Eb et al.) The cells are cultured to allow for homologous recombination to occur. Selection using the neo marker is used to remove cells not having the identifier in their genome. Subsequently, the cells are grown on a medium containing a substrate for HSV-Tk, which procedure removes cells in which the identifier is integrated randomly.

The identifier is detected using a labeled hybridization probe. The presence of the identifier is also confirmed with the use of the well-known PCR technique (Mullis et al.)

Although explained with the use of various illustrative examples and embodiments, the scope of the invention is to be determined by the accompanying claims.

1. A method for identifying by way of a nucleic acid sequence or an amino acid sequence, a person or entity associated with a biological material, said method comprising:
incorporating an identifier which is a unique combination of building blocks in said nucleic acid sequence or an amino acid sequence, said unique combination of building blocks particularly identifying the person or entity associated with the biological material.

2. The method according to claim 1, wherein said building blocks comprise amino acids or nucleotides/nucleosides.

3. The method according to claim 1, wherein said identifier encodes the trade name or a trademark of person or entity associated with the biological material.

4. The method according to claim 1 wherein said identifier comprises the trade name and/or trademark of the person or entity associated with the biological material in amino acids and/or amino acid encoding codons of nucleotides/nucleosides.

5. A nucleic acid sequence comprising an identifier, said identifier comprising a selected combination of nucleotides/nucleosides, said selected combination of nucleotides/nucleosides identifying a person or entity associated with the nucleic acid sequence.

6. The nucleic acid sequence of claim 5, wherein said selected combination comprises a contiguous sequence of nucleosides/nucleotides.

7. The nucleic acid sequence of claim 5, wherein said selected combination is unique and corresponds to a trade name and/or trademark of the person or entity associated with the nucleic acid sequence.

8. The nucleic acid sequence of claim 5, claim 6, or claim 7, wherein said selected combination is linked, at least in part, to the person or entity's trade name and/or trademark when expressed as single letter amino acid codons.

9. The nucleic acid sequence of any one of claim 5, wherein said identifier is essentially free of nuclease susceptibility.

10. A cell comprising the nucleic acid sequence of claim 5, claim 6, claim 7, claim 8 or claim 9.

11. The cell of claim 10, wherein said nucleic acid sequence is integrated into the cell's genome and/or the genome of at least one of the cell's organelles.

12. A microorganism comprising the cell of claim 10 or claim 11.

13. A polypeptide comprising an identifier, said identifier comprising a unique combination of amino acids representative of a person associated with the polypeptide.

14. The polypeptide of claim 13, wherein said identifier is essentially free of protease susceptibility.

15. A method for determining the origin of a biological material, said method comprising:
subjecting said biological material to an assay capable of determining the presence of an identifier of a person or entity associated with the biological material,
wherein said identifier has been incorporated into said biological material.

16. The method according to claim 15, wherein said identifier is a nucleic acid sequence and wherein said assay comprises at least one probe or primer capable of hybridizing to said nucleic acid sequence.

17. The method according to claim 16, wherein said assay comprises a nucleic acid amplification method.

18. A plasmid of the type including a nucleic acid sequence of interest, the improvement comprising:
choosing an indicator of source of origin to identify an owner or originator of the plasmid, and
incorporating the indicator of source of origin into the plasmid in such a way as not to interfere with the nucleic acid sequence of interest.

19. The plasmid of claim 18 wherein the indicator of source of origin is a nucleic acid sequence encoding a peptide having a single letter code sequence spelling a name.

20. The plasmid of claim 19 wherein the nucleic acid sequence encoding a peptide having a single letter code sequence spelling a name is not configured for expression.

21. A cell comprising the plasmid of claim 18.

22. A non-human transgenic organism containing the cell of claim 21.

23. The non-human transgenic organism of claim 22 wherein the non-human transgenic organism is a plant or animal.

24. The plasmid of claim 18 wherein the indicator of origin is a nucleic acid sequence encoding a name using codons encoding a letter of the alphabet or a number.

25. A method of marking a biological polymer comprising monomers, the method comprising:
including as a portion of the monomers a series of monomers encoding a source of origin.

26. The method according to claim 25 wherein the biological polymer is selected from the group consisting of DNA, RNA, polysaccharide, and polypeptide.

27. The method according to claim 25 wherein the biological polymer is a nucleotide sequence, and the monomers are nucleic acids.

28. A method of identifying a first biological polymer, said method comprising:
incorporating into said first biological polymer an indicator of source of origin comprising a second biological polymer encoding the source of origin;
analyzing the first biological polymer to determine the first biological polymer's sequence, including the sequence of the second biological polymer; and
reading the sequence of the second biological polymer to determine the source of origin.

29. The method according to claim 28 wherein the first biological polymer is selected from the group consisting of DNA, RNA, and polypeptide.

30. The method according to claim 28 wherein the second biological polymer encodes the source of origin of the first biological polymer by corresponding monomers in the polymer to a letter of the alphabet or a number.

31. A method for marking a biological polymer with a source of origin, said method comprising:
determining a code of monomers, said monomers being of biological origin, wherein at least one monomer corresponds to at least one alphanumeric character;
translating an indicator of source of origin for an entity into a series of said monomers; and
incorporating said series of monomers into a biological polymer made by or for said entity.

32. The method according to claim 31 wherein the source of origin encodes the entity's name.
